(19) United States
(12) Patent Application Publication
(10) Pub. No.: US 2020/0368058 A1
(43) Pub. Date: Nov. 26, 2020

(54) CATHETER COLLECTION SYSTEM

(71) Applicant: Sarah Snider, Ankeny, IA (US)

(72) Inventor: Sarah Snider, Ankeny, IA (US)

(21) Appl. No.: 16/877,600

(22) Filed: May 19, 2020

Related U.S. Application Data
(60) Provisional application No. 62/850,003, filed on May 20, 2019.

(51) Int. Cl. A61F 5/451; A61F 5/44; A61M 25/00

(52) U.S. Cl. CPC A61F 5/451; A61F 5/4405; A61M 2210/1089; A61M 2210/1085; A61M 25/0017

(57) ABSTRACT

A catheter collection system having a tube with a first end and a second end connected to a collection bag with a connection member. Disposed within the tube is a check valve which permits fluid flow from a patient's bladder to the collection bag and prevents fluid flow from the collection bag back to the patient. The tube is connected to the catheter and attached to the catheter is a stop.

### CROSS REFERENCE TO RELATED APPLICATION

This application claims the benefit of priority from U.S. Provisional Application No. 62/850,003 filed May 20, 2019, the contents of this application is hereby incorporated by reference in its entirety.

### BACKGROUND OF THE INVENTION

This invention is directed to a catheter collection system and more particularly a catheter collection system that is more emotionally sensitive to use and helps to prevent infections.

Catheter collection systems are well-known in the art and typically include a tube connected to a bag. When in use, such as when in a wheelchair, the bag filled partially with urine can be embarrassing to the patient and emotionally painful to use. In addition, through movement, there are times where, due to gravity, urine flows back from the bag and/or tube into the patient causing infection. Accordingly, there is a need in the art for a system that addresses these deficiencies.

An objective of the present invention is to provide a catheter collection system that is less emotionally painful to use.

Another objective of the present invention is to provide a catheter collection system that prevents urine flow back to a patient and reduces the risk of infection.

These and other objectives will be apparent to one skilled in the art based upon the following written description, drawings and claims.

### SUMMARY OF THE INVENTION

A catheter collection system includes a tube having a first end and a second end connected to a collection bag with a connecting member. Disposed within the tube is a check valve which permits fluid flow from the patient to the collection bag and prevents fluid flow bag toward the patient. The collection bag has a drainage opening with a valve associated with the drainage opening.

Connected to the tube is a catheter. Attached to the catheter is a stop which prevents the catheter from sliding too far into a patient's body.

### BRIEF DESCRIPTION OF THE DRAWINGS

FIG. 1 is a side sectional view of a catheter collection system; and

FIG. 2 is a top plan view of a stop.

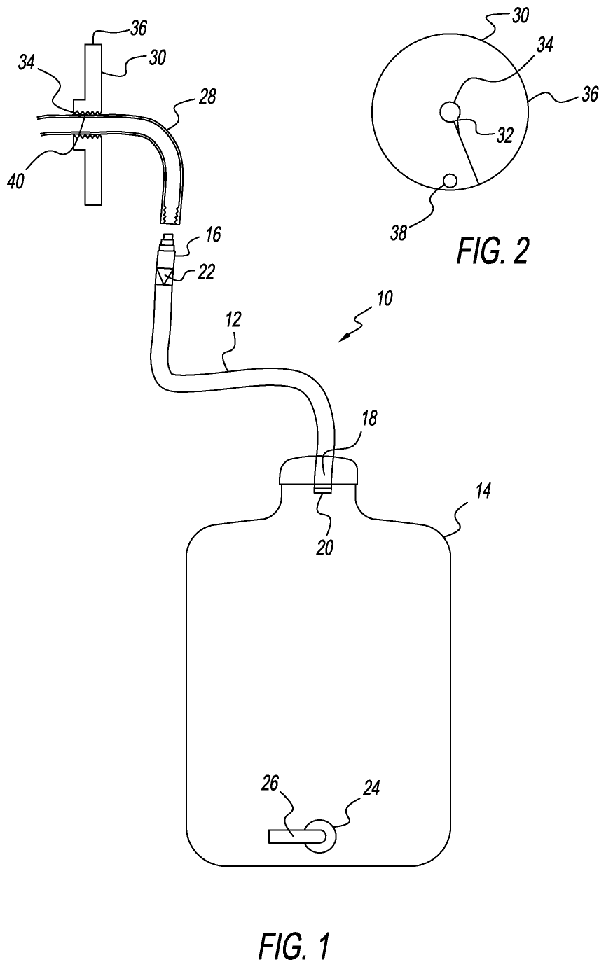

### DETAILED DESCRIPTION

Referring to the Figures, a catheter collection system 10 includes a tube 12 connected to and in communication with a collection bag 14. The collection tube 12 has a first end 16 adapted for insertion into a patient's urethra. The second end 18 of the tube 12 has a connection member 20 adapted to provide a sealed connection between the tube 12 and the bag 14.

Disposed within the tube 12 between the first end 16 and the second end 18 is a check valve 22 that permits fluid flow from the patient's bladder to the bag 14 and prevents fluid flow back toward the patient's urethra. Preferably, the check valve 22 is positioned adjacent the first end 16 of the tube 12 to best prevent the flow of urine back toward the patient which causes infection. The bag 14 also has a drainage opening 24 with a valve 26.

The tube 12 and bag 14 are made of any material and preferably are made of a heat sensitive material adapted to change color or turn opaque when filled with urine. Alternatively, the tube 12 and bag 14 are covered with a decorative design. The shape of the bag 14 can be adapted to match the shape of a toy such as a car or a cartoon character primarily for use by children.

A catheter 28 is attached to the first end 16 of tube 12. The catheter 28 is then inserted into a patient and attached to their pubic bone or urethra. Attached to the catheter 28 is a stop 30. While the stop 30 is of any size, shape, and structure, in one example, the stop 30 is circular having a cut-out section 32 that extends from a central opening 34 to an outer perimeter 36. Adjacent the cut-out section 32 is a connecting member 38 such as a snap or the like. The central opening 34 has an inner surface having a plurality of gripping ridges teeth 40 to prevent the stop 30 from sliding in relation to the catheter 28.

Preventing the catheter 28 from being pushed too far into the patient is important because if pushed in too far the catheter 28 won't drain. Clothing as well as simple movement can provide a pushing force on the catheter 28.

When used, the bag 14 is attached to a bed, a wheelchair, or a patient. Alternatively, the bag 14 is securely received within a transport member such as a backpack or the like.

In operation, once the tube 12 is connected to the patient, urine will flow from the patient, through the tube 12, and into the bag 14. The check valve 22 prevents urine from flowing from the tube 12 back into the patient's body causing infection. As a result of the check valve 22, the bag 14 is not required to be positioned below the tube 12.

From the above discussion and accompanying figures and claims it will be appreciated that a catheter collection system 10 offers many advantages over the prior art. It will be appreciated further by those skilled in the art that other various modifications could be made to the device without parting from the spirit and scope of this invention. All such modifications and changes fall within the scope of the claims and are intended to be covered thereby. It should be understood that the examples and embodiments described herein are for illustrative purposes only and that various modifications or changes in the light thereof will be suggested to persons skilled in the art and are to be included in the spirit and purview of this application.

What is claimed is:

1. A catheter collection system, comprising:
a tube having a first end and a second end,
a collection bag connected to the tube at the second end of the tube,
a check valve disposed within the tube and adapted to permit fluid flow from a patient's bladder to the collection bag and prevents fluid flow back toward the patient.

2. The system of claim 1 wherein the check valve is positioned adjacent the first end of the tube.

3. The system of claim 1 wherein the collection bag is connected to the tube using a connection member.

4. The system of claim 1 wherein the collection bag has a drainage opening and a valve associated with the drainage opening.

5. The system of claim 1 wherein a catheter is connected to the first end of the tube.

6. The system of claim 5 wherein a stop is connected to the catheter.

7. The system of claim 6 wherein the stop has a cut-out section that extends from a central opening to an outer perimeter and a connecting member adjacent the cut-out section.

8. The system of claim 7 wherein the central opening has a plurality of gripping teeth.

9. The system of claim 1 wherein the tube and the collection bag are made of a heat sensitive material.

10. The system of claim 1 wherein the collection bag has a decorative shape.
